SEQUENCE LISTING (33 sequences)

SEQ ID NO: 1 - DNA, 1003 bp, human
tcagtaatgccttttttggtcaccttatataaaattgctccccattttccatctctaaactctcctttcttctttcctgccttctttttttattaggccttatcacctcacacataatacattgtgttttctctttctttacttttttagtgtatgaattcctgcaaaaccatgtattaaataaaatttttgtgtgctttgttcgctgttatttttctaacatctagcattgtgtctggcatacaataatgctcaatgaatgttttttgaatgaaaaaattgattaaatggatgcatgaattaacaaatgttagtttattctgtatacttactccttgattttgaatttttatacaatggttgctaaatccagtaaggtcatagttcgttctttagatcattcagtgttggactcactctgcaccagtgtcagatgacctaattacatgttcaggagggaggccatgactcgaagaatgcacagcctgagttacaccggatggtctttggatcaggctgctctaccctgattattcccctagggggagacagaggtctaagcactctgtaagtgtatgactcctagaatctatgaaaagagcactgcagatttcaggaaggctggttatggggcatctccaacctgtcataggagctggtaattatggagacactataccctacatgtaagaggatgcctggaagagaagttgcctggagcatatttaacatgagagactcgaattgaaacctgtttagccagaaccaatgatttgaattcacaacctttccaaagggcccctggctgtgttgttgattctccagtggtttgtgtcccaacgtttcctggcattacctaacctggattctggttgacagctcctgattggtgccctctgcatatatattgtcaggatgtggaatcctgaagtcagcgccttgccttctcttaggctttgaagcatttttgtctgtgctccctgatcttcaggtcaccaccatg

SEQ ID NO: 2 - DNA, 27 bp, human
ctggttgacagctcctgattggtgccc

SEQ ID NO: 3 - DNA, 66 bp, human
ctggttgacagctcctgattggtgccctctgcatatatattgtcaggatgtggaatcctgaagtca

SEQ ID NO: 4 - DNA, 64 bp, human
ctggttgacagctcctgattggtgccctctgcatatatattgtcaggatgtggaatcctgaagt

SEQ ID NO: 5 - DNA, 132 bp, human
ctggttgacagctcctgattggtgccctctgcatatatattgtcaggatgtggaatcctgaagtcagcgccttgccttctcttaggctttgaagcatttttgtctgtgctccctgatcttcaggtcaccacc

SEQ ID NO: 6 - DNA, 88 bp, human
ctgtcataggagctggtaattatggagacactataccctacatgtaagaggatgcctggaagagaagttgcctggagcatatttaaca

SEQ ID NO: 7 - DNA, 252 bp, human
ctgtcataggagctggtaattatggagacactataccctacatgtaagaggatgcctggaagagaagttgcctggagcatatttaacatgagagactcgaattgaaacctgtttagccagaaccaatgatttgaattcacaacctttccaaagggcccctggctgtgttgttgattctccagtggtttgtgtcccaacgtttcctggcattacctaacctggattctggttgacagctcctgattggtgccc

SEQ ID NO: 8 - DNA, 291 bp, human
ctgtcataggagctggtaattatggagacactataccctacatgtaagaggatgcctggaagagaagttgcctggagcatatttaacatgagagactcgaattgaaacctgtttagccagaaccaatgatttgaattcacaacctttccaaagggcccctggctgtgttgttgattctccagtggtttgtgtcccaacgtttcctggcattacctaacctggattctggttgacagctcctgattggtgccctctgcatatatattgtcaggatgtggaatcctgaagtca

SEQ ID NO: 9 - DNA, 289 bp, human
ctgtcataggagctggtaattatggagacactataccctacatgtaagaggatgcctggaagagaagttgcctggagcatatttaacatgagagactcgaattgaaacctgtttagccagaaccaatgatttgaattcacaacctttccaaagggcccctggctgtgttgttgattctccagtggtttgtgtcccaacgtttcctggcattacctaacctggattctggttgacagctcctgattggtgccctctgcatatatattgtcaggatgtggaatcctgaagt

SEQ ID NO: 10 - DNA, 357 bp, human
ctgtcataggagctggtaattatggagacactataccctacatgtaagaggatgcctggaagagaagttgcctggagcatatttaacatgagagactcgaattgaaacctgtttagccagaaccaatgatttgaattcacaacctttccaaagggcccctggctgtgttgttgattctccagtggtttgtgtcccaacgtttcctggcattacctaacctggattctggttgacagctcctgattggtgccctctgcatatatattgtcaggatgtggaatcctgaagtcagcgccttgccttctcttaggctttgaagcatttttgtctgtgctccctgatcttcaggtcaccacc

SEQ ID NO: 11 - DNA, 173 bp, human
cgaagaatgcacagcctgagttacaccggatggtctttggatcaggctgctctaccctgattattcccctagggggagacagaggtctaagcactctgtaagtgtatgactcctagaatctatgaaaagagcactgcagatttcaggaaggctggttatggggcatctccaac

SEQ ID NO: 12 - DNA, 21 bp, human
gatcttcaggtcaccaccatg

SEQ ID NO: 13 - DNA, 20 bp, human
gggacacactctaccattcg

SEQ ID NO: 14 - DNA, 19 bp, human
acccactcctccacctttg

SEQ ID NO: 15 - DNA, 19 bp, human
ctcttgtgctcttgctggg

SEQ ID NO: 16 - DNA, 21 bp, human
cagaagactcaagctgattcc

SEQ ID NO: 17 - DNA, 21 bp, human
tctttacgagcagtggtagaa

SEQ ID NO: 18 - DNA, 22 bp, artificial
  PCR primer
ctaatacgactcactatagggc

SEQ ID NO: 19 - DNA, 23 bp, artificial
  PCR primer
aagcagtggtatcaacgcagagt

SEQ ID NO: 20 - DNA, 26 bp, human
gctccccattttccatctcgagcatc

SEQ ID NO: 21 - DNA, 26 bp, human
caatggttgctaactcgagtaaggtc

SEQ ID NO: 22 - DNA, 24 bp, human
agggaggccatgactcgaggaatg

SEQ ID NO: 23 - DNA, 23 bp, human
ctccaactcgagataggagctgg

SEQ ID NO: 24 - DNA, 26 bp, human
ggagcatatttaactcgagagactcg

SEQ ID NO: 25 - DNA, 25 bp, human
ctccagtggctcgagtcccaacgtt

SEQ ID NO: 26 - DNA, 26 bp, human
taacctggatctcgagtgacagctcc

SEQ ID NO: 27 - DNA, 31 bp, human
cctgattggtgcctcgagcatatatattgtc

SEQ ID NO: 28 - DNA, 28 bp, human
cttcaaagcctaagcttaggcaaggcgc

SEQ ID NO: 29 - DNA, 34 bp, artificial
  PCR primer
catatttaacatatgagactccaattgaaacctg

SEQ ID NO: 30 - DNA, 28 bp, artificial
  PCR primer
ggggcatctcccaacatatgataggagc

SEQ ID NO: 31 - DNA, 38 bp, artificial
  PCR primer
acacacacacggtacctcataggagctggtaattatgg

SEQ ID NO: 32 - DNA, 42 bp, artificial
  PCR primer
gaagttgcctggagcatatttaacctcgagggttggttggtt

SEQ ID NO: 33 - DNA, 33 bp, artificial
  PCR primer
gttgcctggtctaatgtaaacatgagagactcg

(19) United States
(12) Patent Application Publication
(10) Pub. No.: US 2009/0216005 A1
(43) Pub. Date: Aug. 27, 2009

(54) Small Breast Epithelial Mucin Gene Promoter

(76) Inventors: Hube Florent, Winnipeg (CA); Yvonne Myal, Winnipeg (CA); Etienne Leygue, Winnipeg (CA)

Correspondence Address: ADE & COMPANY INC., 2157 Henderson Highway, WINNIPEG, MB, R2G1P9, CA

(21) Appl. No.: 11/718,768

(22) PCT Filed: Nov. 8, 2005
(86) PCT No.: PCT/CA05/01697

§ 371 Date: Oct. 24, 2007

Related U.S. Application Data
(60) Provisional application No. 60/625,595, filed on Nov. 8, 2004.

(51) Int. Cl. C12N 15/11

(52) U.S. Cl. 536/23.5

(57) ABSTRACT

The human small breast epithelial mucin gene promoter is analyzed and regions corresponding to a minimum promoter element for breast cell-specific expression, an enhancer and a negative regulatory element are identified.

### PRIOR APPLICATION INFORMATION

This application claims the benefit of U.S. Ser. No. 60/625,595, filed Nov. 8, 2004.

### FIELD OF THE INVENTION

The present Invention relates generally to the field of promoter elements.

### BACKGROUND OF THE INVENTION

Breast cancer, with an estimated 1 million new cases every year, remains the most common type of cancer in women (23% of all cancers) in developed countries (Parkin et al., 2005, CA Cancer J Clin 55: 74-108). Breast tumor progression, a multistep process beginning with a benign stage and progressing through hyperproliferation of the breast epithelium to invasive carcinoma, is characterized by multiple changes in gene expression (Wellings and Jensen, 1973, J Natl Cancer Inst 50: 1111-1118; Krishnamurthy and Sneige, 2002, Adv Anat Pathol 9: 185-197; Schmidt, 2002, Am J Pathol 161: 1973-1977; Garnis et al., 2004, Mol Cancer 3: 9). It has been suggested that the identification of genes differentially expressed during the transition from a normal to a cancer cell, together with the elucidation of the mechanisms controlling their expression, will lead to the establishment of novel diagnostic and therapeutic strategies for the clinical management of this disease.

We previously identified a new gene with a tissue-restricted expression. SBEM (small breast epithelial mucin) is indeed strongly expressed in normal and tumoral mammary and salivary gland, whereas others tissues were negative for SBEM expression.

Clearly, identification of a breast specific sequence could facilitate the targeting of breast cancer cells for therapeutic gene delivery. It is therefore desirable to study hSBEM promoter regions to identify sequences responsible for such breast specific expression of this gene.

### SUMMARY OF THE INVENTION

According to a first aspect of the invention, there is provided a nucleic acid molecule having at least 70% homology to SEQ ID No. 2.

### BRIEF DESCRIPTION OF THE DRAWINGS

FIG. 1 shows the sequence of the SBEM promoter element (SEQ ID NO. 1).

FIG. 2 is a schematic diagram of the SBEM gene, mRNA and protein structures.

FIG. 3 shows the results of RACE cloning of the SBEM cDNA ends.

FIG. 4 shows the two transcription initiation sites for the SBEM mRNA.

FIG. 5 shows gene expression from a number of upstream deletions of the SBEM promoter region. A. Different constructs used in luciferase assay are numbered from P1 (longer construct) to P8 (smaller construct). Partial 5′-deleted SBEM promoter regions were cloned in pGL3-basic containing the luciferase reporter gene as discussed below. Number on the left indicates positions in the promoter sequence relative to the start site (ATG). The gray box corresponds to the putative TATA-boxes. B. Promoter activities of the SBEM promoter deletion constructs transiently transfected in MCF-7, BT-20, HeLa and HepG2 cells. Luciferase activity, shown as n-fold value compared to cells transfected with the promoterless pGL3-basic vector, was measured as indicated below. Data represent the means of at least three independent transfection experiments.

FIG. 6 shows the effect of deletion and insertion of the 87 by enhancer region on gene expression. Activity of mutated P1 constructs used in luciferase assay. A. P1 modified constructs were cloned in pGL3-basic containing the luciferase reporter gene as discussed below. P1 plus (P1+) contains two copies of the ENH region, and P1 minus (P1−) does not contain any. The gray box corresponds to the putative TATA-boxes and ENH region is in black. B. Promoter activities of the SBEM mutated P1 constructs transiently transfected in MCF-7, BT-20, HeLa and HepG2 cells. Luciferase activity, shown as n-fold value compared to cells transfected by the promoterless pGL3-basic vector, was measured as indicated below. Stars indicate activities that are statistically different (t-test; p<0.05) from activity of P1 minus. Data represent the means of at least three independent transfection experiments.

FIG. 7 shows, the effect of adding the 87 by enhancer element to SBEM promoter constructs lacking the negative regulatory region. Activity of mutated P4 constructs used in luciferase assay. A. P4 modified constructs were cloned in pGL3-basic containing the luciferase gene as described below. P4plus contains two copies of the ENH region, and P5 does not contain any. The gray box corresponds to the putative TATA-boxes and ENH region is in black. B. Promoter activities of the SBEM mutated P4 constructs transiently transfected in MCF-7, BT-20, HeLa and HepG2 cells. Luciferase activity, shown as n-fold value compared to cells transfected by the promoterless pGL3-basic vector, was measured as indicated below. Stars indicate activities that are statistically different (t-test; p<0.05) from activity of P5. Data represent the means of at least three independent transfection experiments.

FIG. 8. RT-PCR analysis of SBEM gene expression in 2 mammary and 2 non-mammary cancer cell lines. Total RNA was extracted from human breast cancer cells (MCF-7 and BT-20) and non-breast cancer cells (HeLa and HepG2), reverse-transcribed and PCR amplified with SBEM or GAPDH primers, as discussed below and in Table 1. Lane M: PhiX174 RF DNA/Hae III DNA ladder. Data are representative of at least two independent RNA extractions for each cell lines.

FIG. 9. Nucleotide sequence of the SBEM promoter. The coding region (underlined) starts at the ATG (+1) and non-coding sequence is in uppercase non-underlined. The overlapping TATA-boxes and the two transcription initiation sites are boxed in gray. Promoter fragments used are shown (P1 to P8), starting in 5′ by arrows and finishing in 3′ by the vertical line (−51). Consensus binding sites for transcription factors in the 87-bp region are shown below the promoter sequence. Nucleotides indicated corresponded to the MatInspector matrix used, with the core sequences boxed in black. Nkx2-5, NK2 transcription factor related; AIRE, Autoimmune regulator; Oct, binding site for octamer-binding transcription factor.

FIG. 10. Activity of P4 and P40M constructs used in luciferase assay. A. P40M was mutated in the octamer-binding site located in (−282/−274) as compared to the wild-type construct P4. The gray box corresponds to the putative TATA-boxes and ENH region is in black. B. Promoter activities of the SBEM P4 and P40M constructs transiently transfected in MCF-7, BT-20, HeLa and HepG2 cells. Luciferase activity, shown as n-fold value compared to cells transfected by the promoterless pGL3-basic vector, was measured as discussed below. Data represent the means of three independent experiments.

FIG. 11. Up-regulation of exogenous and endogenous SBEM promoter activities. A. Promoter activities of the SBEM P4 constructs co-transfected with expression vectors pOct1, pOct2 and the corresponding empty vector peOct in MCF-7, BT-20, HeLa and HepG2 cells. Luciferase activity, shown as n-fold value compared to cells transfected by the promoterless pGL3-basic vector, was measured as discussed below. Data represent the means of at least three independent transfection experiments. B. RT-PCR analysis of mRNA extracted from human breast cancer cells (MCF-7 and BT-20) and non-breast cancer cells (HeLa and HepG2) formerly transfected with peOct, pOct1, and pOct2. Total RNA was reverse-transcribed and PCR amplified with SBEM or GAPDH primers as discussed below and in Table 1. Data are representative of two independent RNA extraction for each cell lines.

### DESCRIPTION OF THE PREFERRED EMBODIMENTS

Unless defined otherwise, all technical and scientific terms used herein have the same meaning as commonly understood by one of ordinary skill in the art to which the invention belongs. Although any methods and materials similar or equivalent to those described herein can be used in the practice or testing of the present invention, the preferred methods and materials are now described. All publications mentioned hereunder are incorporated herein by reference.

As used herein, “purified” does not require absolute purity but is instead intended as a relative definition. For example, purification of starting material or natural material to at least one order of magnitude, preferably two or three orders of magnitude is expressly contemplated as falling within the definition of “purified”.

As used herein, the term “isolated” requires that the material be removed from its original environment.

In this context, a gene called SBEM (small breast epithelial mucin, for review, see Hube et al., 2004, DNA Cell Biol 23: 842-849), preferentially expressed in breast epithelial cells, and over-expressed in breast tumor has been identified (Houghton et al., 2001, Mol Diagn 6: 79-91; Miksicek et al., 2002, Cancer Res 62: 2736-2740; Colpitts et al., 2002, Tumor Biol 23: 263-278). Dot blot analyses revealed a strong SBEM gene expression in breast tissues and salivary glands, whereas all other normal tissues such as brain, ovary, uterus, prostate, or lung were negative (Miksicek et al., 2002; Colpitts et al., 2002). This apparent breast-specific expression was further established by RT-PCR as most of the mammary cancer cell lines tested expressed SBEM transcript (7 out of 8), as opposed to none out of the 10 non-breast tumor cell lines examined (Miksicek et al., 2002). SBEM mRNA expression, widely detectable throughout breast tumorigenesis and tumor progression with more than 90% of primary and metastatic breast tumors expressing this transcript, is increased in tumor compared to normal breast tissues (Houghton et al., 2001; Colpitts et al., 2002). Altogether, data accumulated suggest that SBEM, one of the most “breast-specific” genes described to date, is a breast tumor biomarker and breast-specific target for future therapeutic strategies.

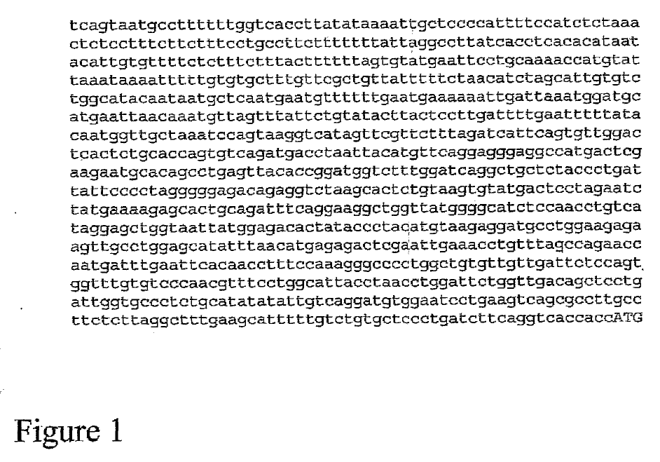

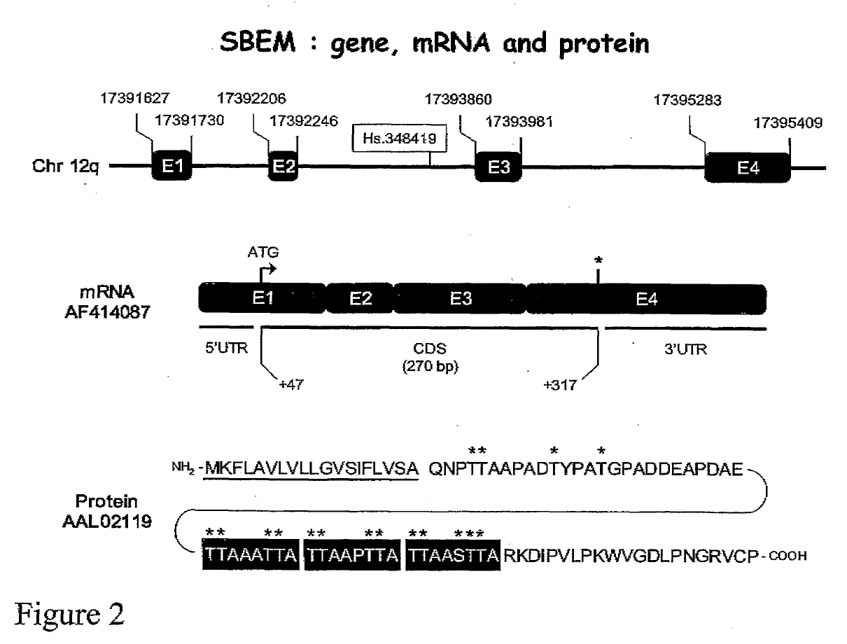

We recently identified a new highly breast-specific gene that we have termed human small breast epithelial mucin (SBEM) (shown in FIG. 2). To elucidate the molecular mechanisms underlying the tissue-specific transcriptional regulation of SBEM, we studied the SBEM gene promoter. A 1-kb human SBEM gene 5′-flanking region was isolated, cloned and sequenced (shown In FIG. 1, SEQ ID NO. 1). The promoter region contains two typical mammalian overlapping TATA boxes, indicating the presence of two transcription initiation sites. Subsequently, these sites were experimentally located by 5′-RACE PCR to −69 and −67 upstream from the translation initiation site, as discussed below. Several transcription factor binding sites (DATA, Oct, NAY, RFX) were also identified, some of which were clustered in a “hormone response region” (half binding-sites for estrogen receptor, Sox9, RORalpha). To identify regions responsible for the breast-specificity of SBEM expression, a series of luciferase reporters driven by different size fragments from the SEEM promoter were constructed, as discussed below. In addition of the minimal promoter located in the −1321-106 region, we identified an 87 by sequence (−357/−270) which is responsible for strong expression in mammary cancer cells only. As will be apparent to one of skill in the art, identification of this breast specific sequence could facilitate the targeting of breast cancer cells for therapeutic gene delivery.

As discussed herein, the isolation of a breast cell-specific promoter is described herein. Also described are the transcription initiation site, putative TATA-boxes, an enhancer region and minimal promoter element, as discussed below.

In one embodiment of the invention, there is provided an isolated or purified nucleic acid molecule having a DNA sequence that consists of:

(SEQ ID No. 1)

tcagtaatgc cttttttggt caccttatat aaaattgctc
cccattttcc atctctaaac tctcctttct tctttcctgc
cttctttttt tattaggcct tatcacctca cacataatac
attgtgtttt ctctttcttt acttttttag tgtatgaatt
cctgcaaaac catgtattaa ataaaatttt tgtgtgcttt
gttcgctgtt atttttctaa catctagcat tgtgtctggc
atacaataat gctcaatgaa tgttttttga atgaaaaaat
tgattaaatg gatgcatgaa ttaacaaatg ttagtttatt
ctgtatactt actccttgat tttgaatttt tatacaatgg
ttgctaaatc cagtaaggtc atagttcgtt ctttagatca
ttcagtgttg gactcactct gcaccagtgt cagatgacct
aattacatgt tcaggaggga ggccatgact cgaagaatgc
acagcctgag ttacaccgga tggtctttgg atcaggctgc
tctaccctga ttattcccct agggggagac agaggtctaa
gcactctgta agtgtatgac tcctagaatc tatgaaaaga
gcactgcaga tttcaggaag gctggttatg gggcatctcc
aacctgtcat aggagctggt aattatggag acactatacc
ctacatgtaa gaggatgcct ggaagagaag ttgcctggag
catatttaac atgagagact cgaattgaaa cctgtttagc
cagaaccaat gatttgaatt cacaaccttt ccaaagggcc
cctggctgtg ttgttgattc tccagtggtt tgtgtcccaa
cgtttcctgg cattacctaa cctggattct ggttgacagc
tcctgattgg tgccctctgc atatatattg tcaggatgtg
gaatcctgaa gtcagcgcct tgccttctct taggctttga
agcatttttg tctgtgctcc ctgatcttca ggtcaccacc
ATG.

As will be appreciated by one of skill in the art, the SBEM promoter element may be fused or otherwise operably linked to a chimeric or non-native gene as discussed below for driving expression of the non-native gene preferentially in breast cells.

As discussed herein and as will be apparent to one of skill in the art, there are likely genetic variants of the SBEM promoter which contain variations or differences in sequence compared to SEQ ID No. 1, for example, deletions, insertions and substitutions which may not significantly alter breast-cell specific expression from the variant SBEM promoter element. That is, these variations or mutations may not significantly alter the activity of the SBEM promoter. Accordingly, such variants are within the scope of the invention. It is of note that such variants could easily be determined by one of skill in the art by using the SBEM promoter sequence described herein to design suitable primers for amplifying and/or sequencing regions of interest of the SBEM promoter.

It is further noted that the promoter elements as described herein may tolerate insertions, deletions and substitutions that do not significantly alter activity and/or function of the breast cell-specific promoter. Such tolerated mutations and/or alterations can easily be determined using reporter gene constructs such as those described herein and any one of the various protocols for inducing random mutagenesis in a desired sequence known in the art. Accordingly, there is provided an isolated nucleic acid molecule having breast-cell specific promoter activity and comprising or having at least 70% homology to SEQ ID No. 1, or at least 75% homology, or at least 80% homology, or at least 85% homology or at least 90% homology to SEQ ID No. 1 or at least 95% homology to SEQ ID No. 1. That is, the isolated nucleic acid molecule when operably linked to a gene of interest, for example, a reporter gene such as luciferase, or a chimeric or non-native gene as discussed herein, is capable of driving breast cell-specific expression of said gene of interest. It is further noted that many programs for comparison of nucleic acid sequences and for determining percent homology are well known and widely available.

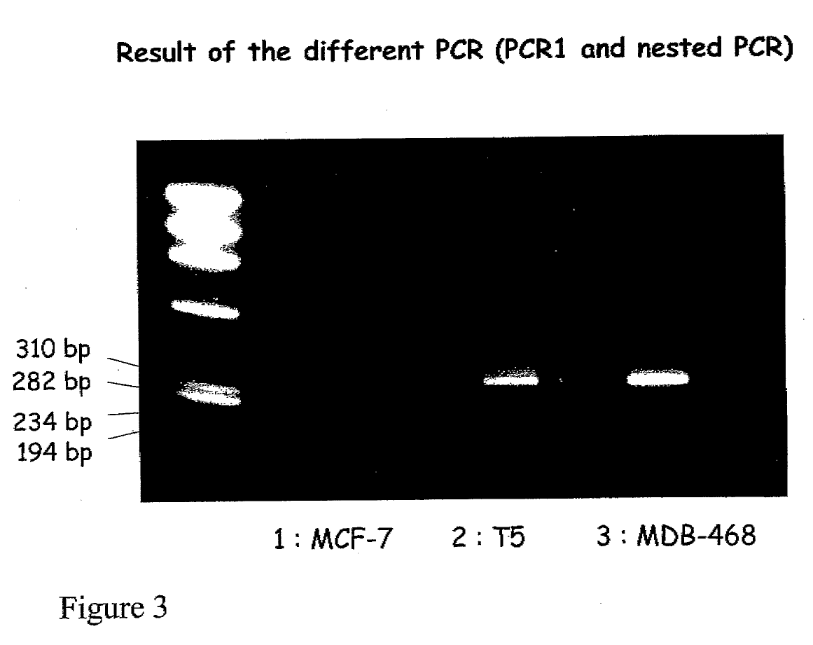

As discussed above, the transcription Initiation sites of SBEM were determined by RACE-PCR (rapid amplification of cDNA ends), as shown in FIG. 3. As is known to those of skill in the art, RACE-PCR involves the steps of: mRNA isolation; Reverse transcription; Adaptor fixation; PCR1; PCR2 (nested PCR) and Cloning and sequencing.

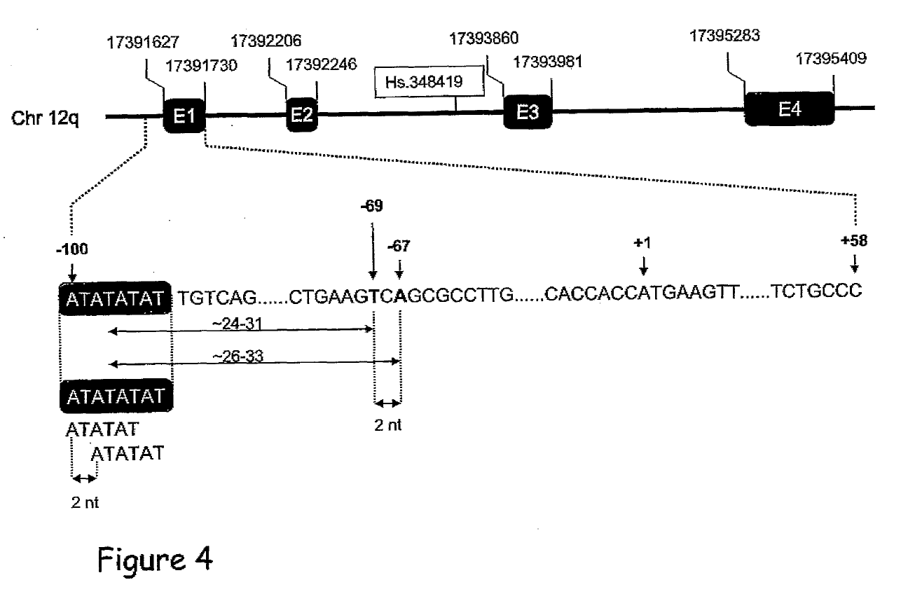

In three breast cell lines MCF-7, T5 and MDB-468, two distinct transcription initiation sites were identified (−69 and −67 from the ATG, as shown in FIG. 4). The frameshift observed (2 bp) is the same as observed in the two overlapped TATA-boxes, located about 30 nucleotides upstream from the transcription initiation sites.

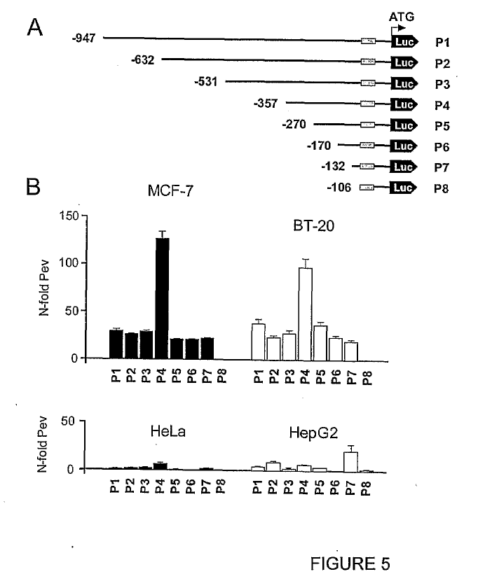

In order to identify the promoter elements required for SBEM expression, constructs were prepared which comprised the luciferase gene fused to the SBEM promoter at the translation start site, shown in FIG. 5. A series of upstream deletion constructs were then prepared and luciferase activity in three breast cell lines: MCF-7, T5 and MDB-468 as, well as two non-breast cell lines, HeLa and HepG2. As can be seen, mammary cell lines expressed a strong luciferase activity, whereas other cell types expressed a low luciferase activity. Furthermore, as can be seen from the results with the deletion constructs in the breast cell lines, only construct #6 (−106) lacked expression, meaning that the minimal promoter region lies between constructs #5 and #6, that is, the minimal promoter region was identified in the −132/−106 region.

Furthermore, the region located between −357 to −270, an 87 bp sequence, is important for a strong SBEM expression and the −531 to −357 region contains a negative regulatory element inhibiting the 87-bp activity, as evidenced by the luciferase activity detected for construct #3 (−357) in the breast cell lines. Furthermore, as can be seen in the luciferase activity for construct #4 (−270), removal of the 87-bp region strongly decreased the SBEM promoter activity.

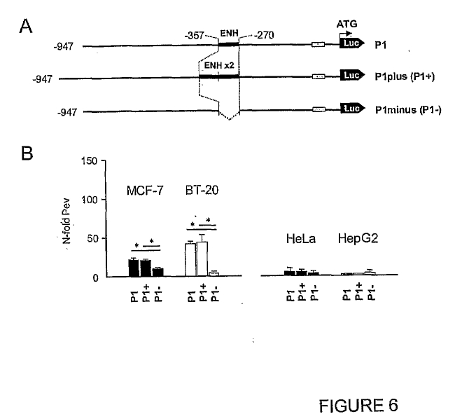

Shown in FIG. 6 is a further set of experiments, wherein constructs were prepared which contained either two copies of the 87 bp region (FIG. 6, #2) or In which the 87 bp region was deleted (FIG. 6, #3). As can be seen, these constructs were made using construct #1 (−947) shown in FIG. 5 which comprises the SBEM promoter fused to the luciferase gene, as discussed above. As can be seen from FIG. 5, addition of one copy of the 87-bp (construct #2) region to the full-length SBEM promoter increased promoter activity in only ZR-75 cells but deletion of the 87 bp region strongly decreased SBEM promoter activity in all breast cell lines (construct #3).

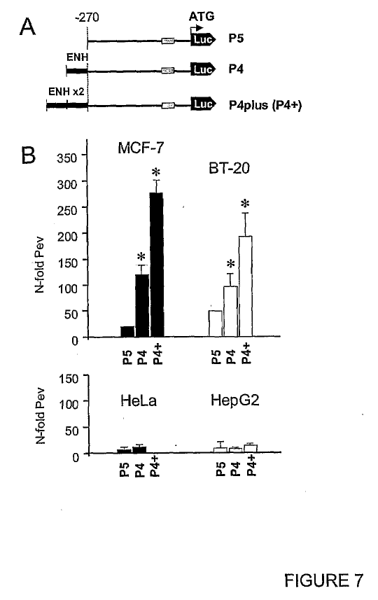

Shown in FIG. 7 are further SBEMp-luciferase constructs in which the negative regulatory region, discussed above, has been deleted and the 87 bp region is deleted (FIG. 7, construct #1) or duplicated (FIG. 7, construct #3). As can be seen, addition of the 87-bp region to the SBEM promoter lacking the negative regulatory region (−947/−357) strongly increased the promoter activity. Furthermore, the enhancing effect of the 87-bp region is only observed in mammary cells, as no effect is seen in HeLa or HepG2 cells.

As discussed above, two distinct transcription initiation sites were identified (−69 and −67 from the ATG). The minimal promoter was identified in −132/−106 region. Mammary cell lines expressed a strong luciferase activity from the SBEMp-luciferase constructs, whereas negative cells for SBEM mRNA expressed a low luciferase activity. The region comprised in −357/−270 (87 bp) is therefore an enhancer necessary for a strong breast-specific SBEM expression.

In another aspect of the invention, there is provided a purified or isolated nucleic acid molecule comprising the minimum promoter element of the SBEM promoter as discussed above. That is, there is provided an isolated or purified nucleic acid molecule having or comprising the following sequence:

| | |
|---|---|
| ct ggttgacagc tcctgattgg tgccc. | (SEQ ID No. 2) |

As will be appreciated by one of skill in the art, the SBEM promoter element may be fused or otherwise operably linked to a chimeric or non-native gene as discussed below for driving expression of the non-native gene preferentially in breast cells, as discussed above. As will be appreciated by one of skill in the art, such a construct may also a chimeric or non-native TATA-box element spaced accordingly downstream of the minimal promoter element, and a leader sequence spaced appropriately downstream of the TATA-box (approximately 30 nucleotides). Both of these elements would of course be upstream of the chimeric or non-native gene. As will be appreciated by one of skill in the art, TATA-boxes and leader sequences are well-known in the art. It is further noted that the TATA-box and the leader sequence do not necessarily need to be derived from the chimeric or non-native gene but may be from a different gene or source.

As discussed herein and as will be apparent to one of skill in the art, there are likely genetic variants of the SBEM promoter which contain variations or differences in sequence compared to SEQ ID No. 2, for example, deletions, insertions and substitutions which may not significantly alter breast-cell specific expression from the variant SBEM promoter element. That is, these variations or mutations may not significantly alter the activity of the SBEM promoter. Accordingly, such variants are within the scope of the invention.

Yet further, there is provided an isolated nucleic acid molecule having breast-cell specific promoter activity and comprising or having at least 70% homology to SEQ ID No. 2, or at least 75% homology, or at least 80% homology, or at least 85% homology or at least 90% homology to SEQ ID No. 2 or at least 95% homology to SEQ ID No. 2, as discussed above.

In another embodiment of the invention, there is provided an isolated or purified nucleic acid molecule having a DNA sequence that consists of:

(SEQ ID No. 3)

ct ggttgacagc tcctgattgg tgccctctgc
atatatattg tcaggatgtg gaatcctgaa gtca
or

(SEQ ID No. 4)

ct ggttgacagc tcctgattgg tgccctctgc
atatatattg tcaggatgtg gaatcctgaa gt

As will be appreciated by one of skill in the art, these sequences comprise a segment of the SBEM promoter comprising the minimal promoter element and the transcription initiation site(s). As will be appreciated by one of skill in the art, either one of these sequences can be fused or operably linked to a leader sequence and a cDNA encoding a gene of interest as discussed above. In some embodiments, the purified or isolated nucleic acid molecule as described herein may be fused to a multiple cloning site for facilitating fusion with leader-containing cDNA molecules.

As discussed herein and as will be apparent to one of skill in the art, there are likely genetic variants of the SBEM promoter which contain variations or differences in sequence compared to SEQ ID No. 3 or 4, for example, deletions, insertions and substitutions which may not significantly alter breast-cell specific expression from the variant SBEM promoter element. That is, these variations or mutations may not significantly alter the activity of the SBEM promoter. Accordingly, such variants are within the scope of the invention.

Yet further, there is provided an isolated nucleic acid molecule having breast-cell specific promoter activity and comprising or having at least 70% homology to SEQ ID No. 3 or 4, or at least 75% homology, or at least 80% homology, or at least 85% homology or at least 90% homology to SEQ ID No. 3 or 4 or at least 95% homology to SEQ ID No. 3 or 4. That is, the isolated nucleic acid molecule when operably linked to a gene of interest, for example, a reporter gene such as luciferase, or a chimeric or non-native gene as discussed herein, is capable of driving breast cell-specific expression of said gene of interest. It is further noted that many programs for comparison of nucleic acid sequences and for determining percent homology are well known and widely available.

In one embodiment of the invention, there is provided an isolated or purified nucleic acid molecule having a DNA sequence that consists of:

(SEQ ID No. 5)

ct ggttgacagc tcctgattgg tgccctctgc atatatattg
tcaggatgtg gaatcctgaa gtcagcgcct tgccttctct
taggctttga agcatttttg tctgtgctcc ctgatcttca
ggtcaccacc

As will be appreciated by one of skill in the art, this sequence comprises a segment of the SBEM promoter comprising the minimal promoter element up to the translation initiation site. As will be appreciated by one of skill in the art, this sequence can be fused or operably linked to a cDNA encoding a gene of interest as discussed above. In some embodiments, the purified or isolated nucleic acid molecule as described herein may be fused to a multiple cloning site for facilitating fusion with a cDNA molecule.

As discussed herein and as will be apparent to one of skill in the art, there are likely genetic variants of the SBEM promoter which contain variations or differences in sequence compared to SEQ ID No. 5, for example, deletions, insertions and substitutions which may not significantly alter breast-cell specific expression from the variant SBEM promoter element. That is, these variations or mutations may not significantly alter the activity of the SBEM promoter. Accordingly, such variants are within the scope of the invention.

Yet further, there is provided an isolated nucleic acid molecule having breast-cell specific promoter activity and comprising or having at least 70% homology to SEQ ID No. 5, or at least 75% homology, or at least 80% homology, or at least 85% homology or at least 90% homology to SEQ ID No. 5 or at least 95% homology to SEQ ID No. 5. That is, the isolated nucleic acid molecule when operably linked to a gene of interest, for example, a reporter gene such as luciferase, or a chimeric or non-native gene as discussed herein, is capable of driving breast cell-specific expression of said gene of interest. It is further noted that many programs for comparison of nucleic acid sequences and for determining percent homology are well known and widely available.

In another embodiment of the invention, there is provided an isolated or purified nucleic acid molecule having a DNA sequence that consists of:

(SEQ ID No. 6)

ctgtcat aggagctggt aattatggag acactatacc
ctacatgtaa gaggatgcct ggaagagaag ttgcctggag
catatttaac a

As will be appreciated by one of skill in the art, this sequence comprises a segment of the SBEM promoter comprising the enhancer or ENH sequence. As will be appreciated by one of skill in the art, this sequence can be fused or operably linked to a chimeric or non-native nucleic acid molecule encoding a promoter, TATA-box, leader sequence and cDNA as discussed above, encoding a gene of interest as discussed above. In some embodiments, the purified or isolated nucleic acid molecule as described herein may be fused to a multiple cloning site for facilitating fusion with a cDNA molecule. As discussed above, this element may be used for increasing expression in breast cells.

As discussed herein and as will be apparent to one of skill in the art, there are likely genetic variants of the SBEM promoter which contain variations or differences in sequence compared to SEQ ID No. 6, for example, deletions, insertions and substitutions which may not significantly alter breast-cell specific expression from the variant SBEM promoter element. That is, these variations or mutations may not significantly alter the activity of the SBEM promoter. Accordingly, such variants are within the scope of the invention.

Yet further, there is provided an isolated nucleic acid molecule having breast-cell specific promoter activity and comprising or having at least 70% homology to SEQ ID No. 6, or at least 75% homology, or at least 80% homology, or at least 85% homology or at least 90% homology to SEQ ID No. 6 or at least 95% homology to SEQ ID No. 6. That is, the isolated nucleic acid molecule when operably linked to a gene of interest, for example, a reporter gene such as luciferase, or a chimeric or non-native gene as discussed herein, is capable of driving breast cell-specific expression of said gene of interest. It is further noted that many programs for comparison of nucleic acid sequences and for determining percent homology are well known and widely available.

In other embodiments, there may be provided a nucleic acid molecule comprising a nucleic acid molecule having at least 70%, at least 75%, at least 80%, at least 85%, at least 90% or at least 95% homology to SEQ ID No. 6 upstream of a nucleic acid molecule having at least 70%, at least 75%, at least 80%, at least 85%, at least 90% or at least 95% homology to SEQ ID No. 2 which is in turn upstream of a nucleic acid molecule encoding a chimeric or non-native gene of interest, as discussed herein. It is of note that the enhancer and the minimal promoter region may be adjacent one another or may be at a distance to one another.

In another embodiment of the invention, there is provided an isolated or purified nucleic acid molecule having a DNA sequence that consists of:

(SEQ ID No. 7)

ctgtcat aggagctggt aattatggag acactatacc
ctacatgtaa gaggatgcct ggaagagaag ttgcctggag
catatttaac atgagagact cgaattgaaa cctgtttagc
cagaaccaat gatttgaatt cacaaccttt ccaaagggcc
cctggctgtg ttgttgattc tccagtggtt tgtgtcccaa
cgtttcctgg cattacctaa cctggattct ggttgacagc
tcctgattgg tgccc.

As will be appreciated by one of skill in the art, this sequence comprises a segment of the SBEM promoter comprising the enhancer or ENH sequence and the minimum promoter sequence. As will be appreciated by one of skill in the art, this sequence can be fused or operably linked to a chimeric or non-native nucleic acid molecule encoding a TATA-box, leader sequence and cDNA as discussed above, encoding a gene of interest as discussed above. In some embodiments, the purified or isolated nucleic acid molecule as described herein may be fused to a multiple cloning site for facilitating fusion with a cDNA molecule.

As discussed herein and as will be apparent to one of skill in the art, there are likely genetic variants of the SBEM promoter which contain variations or differences in sequence compared to SEQ ID No. 7, for example, deletions, insertions and substitutions which may not significantly alter breast-cell specific expression from the variant SBEM promoter element. That is, these variations or mutations may not significantly alter the activity of the SBEM promoter. Accordingly, such variants are within the scope of the invention.

Yet further, there is provided an isolated nucleic acid molecule having breast-cell specific promoter activity and comprising or having at least 70% homology to SEQ ID No. 7, or at least 75% homology, or at least 80% homology, or at least 85% homology or at least 90% homology to SEQ ID No. 7 or at least 95% homology to SEQ ID No. 7. That is, the isolated nucleic acid molecule when operably linked to a gene of interest, for example, a reporter gene such as luciferase, or a chimeric or non-native gene as discussed herein, is capable of driving breast cell-specific expression of said gene of interest. It is further noted that many programs for comparison of nucleic acid sequences and for determining percent homology are well known and widely available.

In one embodiment of the invention, there is provided an isolated or purified nucleic acid molecule having a DNA sequence that consists of:

(SEQ ID No. 8)

ctgtcat aggagctggt aattatggag acactatacc
ctacatgtaa gaggatgcct ggaagagaag ttgcctggag
catatttaac atgagagact cgaattgaaa cctgtttagc
cagaaccaat gatttgaatt cacaaccttt ccaaagggcc
cctggctgtg ttgttgattc tccagtggtt tgtgtcccaa
cgtttcctgg cattacctaa cctggattct ggttgacagc
tcctgattgg tgccctctgc atatatattg tcaggatgtg
gaatcctgaa gtca
or

(SEQ ID No. 9)

ctgtcat aggagctggt aattatggag acactatacc
ctacatgtaa gaggatgcct ggaagagaag ttgcctggag
catatttaac atgagagact cgaattgaaa cctgtttagc
cagaaccaat gatttgaatt cacaaccttt ccaaagggcc
cctggctgtg ttgttgattc tccagtggtt tgtgtcccaa
cgtttcctgg cattacctaa cctggattct ggttgacagc
tcctgattgg tgccctctgc atatatattg tcaggatgtg
gaatcctgaa gt

As will be appreciated by one of skill in the art, these sequences comprise a segment of the SBEM promoter comprising the enhancer or ENH sequence, the minimum promoter sequence and the transcription initiation site(s). As will be appreciated by one of skill in the art, this sequence can be fused or operably linked to a chimeric or non-native nucleic acid molecule encoding a leader sequence and cDNA as discussed above. In some embodiments, the purified or isolated nucleic acid molecule as described herein may be fused to a multiple cloning site for facilitating fusion with a cDNA molecule.

As discussed herein and as will be apparent to one of skill in the art, there are likely genetic variants of the SBEM promoter which contain variations or differences in sequence compared to SEQ ID No. 8 or 9, for example, deletions, insertions and substitutions which may not significantly alter breast-cell specific expression from the variant SBEM promoter element. That is, these variations or mutations may not significantly alter the activity of the SBEM promoter. Accordingly, such variants are within the scope of the invention.

Yet further, there is provided an isolated nucleic acid molecule having breast-cell specific promoter activity and comprising or having at least 70% homology to SEQ ID No. 8 or 9, or at least 75% homology, or at least 80% homology, or at least 85% homology or at least 90% homology to SEQ ID No. 8 or 9 or at least 95% homology to SEQ ID No. 8 or 9. That is, the isolated nucleic acid molecule when operably linked to a gene of interest, for example, a reporter gene such as luciferase, or a chimeric or non-native gene as discussed herein, is capable of driving breast cell-specific expression of said gene of interest. It is further noted that many programs for comparison of nucleic acid sequences and for determining percent homology are well known and widely available.

In one embodiment of the invention, there is provided an isolated or purified nucleic acid molecule having a DNA sequence that consists of:

(SEQ ID No. 10)

ctgtcat aggagctggt aattatggag acactatacc
ctacatgtaa gaggatgcct ggaagagaag ttgcctggag
catatttaac atgagagact cgaattgaaa cctgtttagc
cagaaccaat gatttgaatt cacaaccttt ccaaagggcc
cctggctgtg ttgttgattc tccagtggtt tgtgtcccaa
cgtttcctgg cattacctaa cctggattct ggttgacagc
tcctgattgg tgccctctgc atatatattg tcaggatgtg
gaatcctgaa gtcagcgcct tgccttctct taggctttga
agcatttttg tctgtgctcc ctgatcttca ggtcaccacc.

As will be appreciated by one of skill in the art, this sequence comprises a segment of the SBEM promoter comprising the enhancer or ENH sequence, the minimum promoter sequence, the transcription initiation site and the translation initiation site. As will be appreciated by one of skill in the art, this sequence can be fused or operably linked to a chimeric or non-native nucleic acid molecule encoding a cDNA as discussed above. In some embodiments, the purified or isolated nucleic acid molecule as described herein may be fused to a multiple cloning site for facilitating fusion with a cDNA molecule.

As discussed herein and as will be apparent to one of skill in the art, there are likely genetic variants of the SBEM promoter which contain variations or differences in sequence compared to SEQ ID No. 10, for example, deletions, insertions and substitutions which may not significantly alter breast-cell specific expression from the variant SBEM promoter element. That is, these variations or mutations may not significantly alter the activity of the SBEM promoter. Accordingly, such variants are within the scope of the invention.

Yet further, there is provided an isolated nucleic acid molecule having breast-cell specific promoter activity and comprising or having at least 70% homology to SEQ ID No. 10, or at least 75% homology, or at least 80% homology, or at least 85% homology or at least 90% homology to SEQ ID No. 10 or at least 95% homology to SEQ ID No. 10. That is, the isolated nucleic acid molecule when operably linked to a gene of interest, for example, a reporter gene such as luciferase, or a chimeric or non-native gene as discussed herein, is capable of driving breast cell-specific expression of said gene of interest. It is further noted that many programs for comparison of nucleic acid sequences and for determining percent homology are well known and widely available.

As will be apparent to one of skill in the art and as discussed herein, any one of the above-described promoter elements or combinations thereof can be operably linked to a gene of interest as discussed herein or specific expression of said gene of interest in breast cells. There is therefore provided a method of expressing a gene of interest in breast cells comprising operably linking the gene of interest to a nucleic acid molecule having at least 70% homology, or at least 75% homology, or at least 80% homology or at least 85% homology or at least 90% homology or at least 95% homology to at least one of SEQ ID No. 1, SEQ ID No. 2, SEQ ID No. 3, SEQ ID No. 4, SEQ ID No. 5, SEQ ID No. 6, SEQ ID No. 7, SEQ ID No. 8, SEQ ID No. 9 or SEQ ID No. 10 or where appropriate, combinations thereof, transfecting the nucleic acid molecule into breast cells and exposing the breast cells to conditions suitable for breast cell-specific expression from the SBEM promoter fragment selected from SEQ ID No. 1-10.

In one embodiment of the invention, there is provided an isolated or purified nucleic acid molecule having a DNA sequence that consists of:

(SEQ ID No. 11)

cgaagaatgc acagcctgag ttacaccgga tggtctttgg
atcaggctgc tctaccctga ttattcccct agggggagac
agaggtctaa gcactctgta agtgtatgac tcctagaatc
tatgaaaaga gcactgcaga tttcaggaag gctggttatg
gggcatctcc aac

As will be appreciated by one of skill in the art, this sequence comprises a segment of the SBEM promoter comprising the negative regulatory region. As will be appreciated by one of skill in the art, this sequence can be fused or operably linked to a chimeric or non-native nucleic acid molecule encoding a promoter element and a cDNA as discussed above for reducing expression of that cDNA compared to a control having the same promoter and cDNA construct but lacking the negative regulatory element.

As discussed herein and as will be apparent to one of skill in the art, there are likely genetic variants of the SBEM promoter which contain variations or differences in sequence compared to SEQ ID No. 11, for example, deletions, insertions and substitutions which may not significantly alter breast-cell specific expression from the variant SBEM promoter element. That is, these variations or mutations may not significantly alter the activity of the SBEM promoter. Accordingly, such variants are within the scope of the invention.

Yet further, there is provided an isolated nucleic acid molecule having breast-cell specific promoter activity and comprising or having at least 70% homology to SEQ ID No. 11, or at least 75% homology, or at least 80% homology, or at least 85% homology or at least 90% homology to SEQ ID No. 11 or at least 95% homology to SEQ ID No. 11. That is, the isolated nucleic acid molecule when operably linked to a gene of interest, for example, a reporter gene such as luciferase, or a chimeric or non-native gene as discussed herein, is capable of driving breast cell-specific expression of said gene of interest. It is further noted that many programs for comparison of nucleic acid sequences and for determining percent homology are well known and widely available.

It is further of note that if so desired, SEQ ID No. 11 or an isolated nucleic acid molecule having at least 70%, at least 75%, at least 80%, at least 85%, at least 90% or at least 95% thereto as discussed above may be fused or operably linked to any one of the promoter elements discussed above.

As will be apparent to one of skill in the art, sequences responsible for the breast specific expression of SBEM are useful in gene-specific or anti-cancer therapy. Constructs containing these regulatory regions driving the expression of a gene inducing cell death could be made and artificially introduced in cancer tissues. These constructs, inactive in non-breast cells, could selectively express the cytotoxic gene and thus kill breast cancer cells. As will be appreciated by one of skill in the art, suitable cytotoxic and anti-sense genes are well known in the art. For example, the cytotoxic gene may be a gene which encodes a toxic substance or substrate or may encode an enzyme or similar element which converts an otherwise benign substrate into a toxic substrate. An example of a cytotoxic gene is herpes simplex virus thymidine kinase which can be used in combination with ganciclovir. Alternatively, the cytotoxic gene could comprise anti-sense transcripts driven by the SBEM promoter fragment described above. Suitable targets for antisense therapy include but are by no means limited to bcl-2, c-myc and ras. In yet other embodiments, constructs may be prepared wherein at least the minimal promoter region is fused to a tumor suppressor gene, for example, p53, IL-12 or the like. As discussed above, such a construct may also include the SBEM TATA-box and transcriptional start sites, mRNA leader and enhancer regions. It is noted that other suitable chimeric or non-native genes are well known in the art and are discussed in El-Aneed, 2004, European Journal of Pharmacology 498: 1-8, which is incorporated herein by reference for examples of suitable cytotoxic, antisense and tumor suppressive genes.

Accordingly, there is provided an expression system comprising: SBEM promoter operably linked to a cytotoxic gene. The SBEM promoter may comprise, for example, nucleotides −1 to −357, or nucleotides −270 to −357 fused to a different gene as discussed above.

In the present study, we have investigated the transcriptional activity of the foremost—900 by of the SBEM promoter using sequential 5′-deletion and transfection of both mammary and non-mammary cells. We have identified experimentally an 87-bp enhancer region, which increased the promoter activity in mammary but not in other cell tested.

Three particular motifs were identified within this short sequence using software analysis. The first two overlapping motifs corresponded to binding sites for two-transcription factors, Nkx2-5 and the autoimmune regulator (AIRE). Nkx2-5, which belongs to the NK2 family of homeobox transcription factor, is essential for cardiac development (Brown et al., 2004, J Biol Chem 279: 10659-10669), but its expression has never been detected in breast tissue (Brown et al., 2004). Similarly, the expression of the autoimmune regulator AIRE, a transcription factor previously shown to control the self-reactivity of the T cell repertoire, is mainly restricted to the thymus, with low detectable levels in lymph node, fetal liver and spleen (Su and Anderson, 2004, Curr Opin Immunol 16: 746-752), but not in breast tissue. It was therefore unlikely that these two overlapping sites were participating to the strong activity of the SBEM promoter in breast cells.

The identification of an octamer-binding transcription factors (Oct) motif within the 87-bp enhancer region was, however, of particular interest. Indeed, accordingly to the data gathered from Matinspector programs (based TransFac database), this particular octamer motif is recognized by Oct1 transcription factor. Since Oct2, another octamer-binding factor, was found to bind the same motifs as Oct1 within promoter sequences (Gstaiger et al., 1995, Nature 373: 360-362), it is predictable that Oct2 will also bind the Oct1 consensus motif identified in the 87 bp.

Both factors belong to the POU (Pit-1, Oct1/2 and Unc-86) domain family of transcription factors, which are strongly involved in embryogenesis, organ development and cell-type determination (Chen and Sukumar, 2003, J Mammary Gland Biol Neoplasia 8: 159-175). All octamer-binding transcription factors share in common a bipartite DNA binding domain that is composed of a conserved POU-specific domain and of a POU homeodomain (Herr and Cleary, 1995, Genes Dev 9: 1679-1693). Oct1, ubiquitously expressed, as well as Oct2, previously described as a B-lymphocyte-specific factor, are both expressed in breast cancer cell lines (Sturm et al., 1988, Genes Dev 2: 1582-1599; Latchman, 1996, Int J Biochem Cell Biol 28: 1081-1083; Jin et al., 1999, Int J Cancer 81: 104-112). Furthermore, Oct1 and Oct2 are involved not only in the transcriptional regulation of genes strongly expressed in mammary glands such as beta-casein, Prolactin and cyclin D1 genes (Voss et al., 1991, Genes Dev 5: 1309-1320; Zhao et al., 2002, Biochim Biophys Acta 1577: 27-37; Boulon et al., 2002, Mol Cell Biol 22: 7769-7779; Cicatiello et al., 2004, Mol Cell Biol 24: 7260-7274), but also in the activity of the exogenous MMTV1 (mouse mammary tumor virus) promoter, predominantly active in breast tissue (Kim and Peterson, 1995, J Virol 69: 4717-4726; Kim et al., 1996, Mol Cell Biol 16: 4366-4377). The presence, within the strong enhancer region (87-bp ENH), of an octamer-binding site strongly suggested a possible involvement of these factors in the mechanisms underlying SBEM promoter activity in breast cells. The suppression of the reporter activity following the octamer-binding site mutation supports the hypothesis that this motif, rather than Nkx2-5 or the AIRE motif, indeed regulates SBEM promoter activity. Furthermore, the subsequent increase in both exogenous reporter activity and endogenous SBEM mRNA level following over-expression of either Oct1 or Oct2 transcription factor in MCF-7 and BT-20 cells corroborates this assumption.

It has been widely assumed in the past that the octamer-binding factor Oct2 mediated tissue-specific promoter activity, whereas the ubiquitously expressed Oct1 mediates general promoter activity (Dong et al., 2001, J Clin Endocrinol Metab 86: 2838-2844). However, studies have more recently challenged this dogma, underlying a role of Oct1 in the tissue-specific expression of numerous genes (Dong et al., 2001; Prefontaine et al., 1999, J Biol Chem 274: 26713-26719; Gonzalez and Carlberg, 2002, J Biol Chem 277: 18501-18509; Belikov et al., 2004, Mol Cell Biol 24: 3036-3047). It has now been established that both Oct1 and Oct2 were involved in the regulation of tissue-specific genes such as immunoglobulin genes (Sturm et al., 1988; Prefontaine et al., 1999; Belikov et al., 2004; Fletcher et al., 1987, Cell 51: 773-781; Malone et al., Mol Immunol 37: 321-328). As SBEM gene is one of the most “breast-specific” gene identified to date, it could be proposed that these two transcription factors, in addition to regulating the strong expression of the SBEM gene, could also be responsible for the its breast-specificity of its expression.

However, since the over-expression of either Oct1 or Oct2 alone is not sufficient to restore SBEM gene expression in non-mammary cell lines, it is reasonable to assume that a participation of octamer-binding transcription factors to the breast-specific expression of SBEM, if any, will involve other partners. The need for other molecules, beside octamer-binding transcription factors, to induce tissue specificity has been demonstrated in other models (Inamoto et al., 1997, J Biol Chem 272: 29852-29858; Luo et al., 1998, Mol Cell Biol 18: 3803-3810; Malone and Wall, 2002, J Immunol 168: 3369-3375; Lins et al., 2003, EMBO J. 22: 2188-2198; Inman et al., 2005, Mol Cell Biol 25: 3182-3193). For example, the ubiquitous Oct1 and a B-cell-specific co-activator are both required for the B-lymphocyte-specific expression of immunoglobulins (Luo et al., 1998). Similarly, Runx2, a bone-specific transcription factor belonging to the Runt family, participates to the Oct1 induced osteoblast differentiation and chondrocyte maturation (Komori, 2002, J Cell Biochem 87: 1-8). Interestingly, Runx2 has recently been identified in mammary epithelial cells (Barnes et al., 2003, Cancer Res 63: 2631-2637) and was found to participate to the formation of a complex with Oct1 to subsequently contribute to the expression of the mammary gland-specific beta-casein gene in breast tissues (Inman et al., 2005). We propose that such factors, remaining to be identified, interact with octamer-binding transcription factors to regulate the breast specific expression of SBEM gene.

It should be stressed that two other members of the Oct transcription factors family, Oct3 and Oct11, have also been detected in breast cancer cells and tissues, but not in normal breast tissues (Jin et al., 1999). The over-expression of SBEM during breast tumorigenis (Houghton et al., 2001; Colpitts et al., 2002) could therefore result from a change in expression of these alternative octamer-binding transcription factors.

### EXPERIMENTAL PROCEDURES

Cell Culture—Cell lines were obtained from the American Type Culture Collection and were cultured in DMEM with 5% fetal bovine serum (Life Technologies, Inc., Burlington, ON, Canada) supplemented with 100 units/ml, penicillin, 100 μg/mL streptomycin, 2 mM glutamine (Life Technologies), 15 mM sodium bicarbonate and 2 mM glucose. Cells were grown at 37° C. in an atmosphere of 95% air and 5% CO₂. Confluent cells, which viability was determined by the Trypan blue dye exclusion test, were detached by 0.05% trypsin-0.02% EDTA (Life Technologies), seeded in 24-well plates (Coming Inc., NY, USA) and cultured in complete medium until use.

RNA isolation and reverse transcription-polymerase chain reaction (RT-PCR)— Total RNA was extracted from cells using the perfect RNA Mini Kit (Eppendorf, Hambourg, Germany) according to the manufacturer's instructions and reverse transcribed as previously described (8). Briefly, 2 μg of total RNA were reverse-transcribed for 1 hour at 37° C. in 1× incubation buffer containing 300 μM of each deoxynucleotide triphosphate, 50 ng random hexamers, 12 units of RNase Out and 300 units of MMLV Reverse Transcriptase (Life Technologies). The primers used for SBEM amplification consisted of SBEM-F and SBEM-R and are described in Table 1. PCR were performed as previously (Miksicek et al., 2002), i.e. 1 μL of each reverse transcription mixture was amplified in a final volume of 50 μL, in the presence of 20 mM Tris-HCl (pH 8.4), 50 mM KCl, 1.5 mM MgCl2, 200 μM of each deoxynucleotide triphosphate, 200 ng of each SBEM primer, and 0.5 unit of Taq DNA polymerase. Each PCR consisted of 35 cycles (15 s at 94° C., 15 s at 56° C., and 15 s at 72° C.). Primers for the ubiquitously expressed GAPDH1 gene (GAP-F and GAP-R) are shown in Table 1. To amplify cDNA corresponding to GAPDH, 30 cycles of PCR were used (15 s at 94° C., 15 s at 52° C., and 15 s at 72° C.). PCR products were then separated on a 1.5% agarose gel containing 1 mg/ml ethidium bromide and visualized under UV irradiation using the GelDoc2000/ChemiDoc System (BioRad).

Rapid amplification of the 5′-cDNA end (RACE)—A 5′-rapid amplification of cDNA end (RACE) reaction was performed using the SMART RACE cDNA amplification kit (BD Biosciences Clontech, Palo Alto, Calif., USA) according to the manufacturers instructions.

Briefly, total RNA extracted from breast cancer cells (MCF-7, T5 and MDA-MB-468) by the method described above served as starting material. 5′-RACE PCR was successively performed using two nested reverse primers (RACE-L1 and RACE-L2, see Table 1) specific for the SBEM gene. The amplified fragments were subcloned into pCR4 vector (Life Technologies) and sequenced on both strands.

Identification of transcription factors binding sites within the 87-bp enhancer region (ENH)—Identification of transcription factor binding sites was done with MatInspector v2.2 program based on TransFac database (Quandt et al., 1995, Nucleic Acid Res 23: 4878-4884; Wingender et al., 2001, Nucleic Acid Res 29: 281-283; Hube et al., 2003, Thromb Res 109: 207-215) available online (Matinspector, www.genomatix.de; TransFac, www.gene-regulation.com). All parameters were set to default except for the core similarity (1.00), matrix similarity (Optimized +0.04), and matrix group (Vertebrates).

Plasmids—To characterize and identify potential regulatory regions in the SBEM promoter, various deletion constructs of the 5′-flanking region were generated by PCR using modified primers that contained restriction sites (Table 1). The XhoI and HindIII (Invitrogen) restriction enzyme-digested fragments obtained were subsequently subcloned in the promoterless, enhancerless expression vector pGL3-Basic (Promega, Madison, Wis., USA) upstream of the luciferase reporter gene. Mutated SBEM/luciferase plasmids (P4OM, P1 plus, P1 minus and P4plus) were constructed by directed mutagenesis and PCR strategy using the QuickChange Multi Site-Directed Mutagenesis Kit (Siratagene, La Jolla, Calif., USA). P40M corresponded to the P4 plasmid in which the octamer-binding site GGAGCATATTTAA (−284/−272) (SEQ ID No. 34) was replaced by GGTCTAATGTAAA (SEQ ID No. 35). When appropriate, PCR-mutated fragments were subsequently digested by Nde1 (P1 plus and P1 minus) or KpnI and XhoI (P4plus) restriction enzymes (Life Technologies) and subcloned in parental vectors. Sequences of all promoter constructs were then confirmed by dideoxynucleotide chain-termination sequencing.

The expression plasmids pOct1, pOct2 and the corresponding empty vector peOct (Tanaka and Herr, 1990, Cell 60: 375-386) were kindly provided by Dr. Winshop Herr (Cold Spring Harbor Laboratory).

Transient DNA transfections and luciferase assays—The human SBEM/luciferase reporter constructs were transiently transfected into the mammary and non-mammary cell lines (cells cultured in a 24-well plate until 70-80% confluent) using LipofectAMINE Plus® Reagent (Life Technologies) and following the manufacturer's instructions. Briefly, 1.33 nM of appropriate plasmid was mixed with 8 g of LipofectAMINE for each cell lines in a final volume of 200 μL of complete media without PBS. MCF-7 and HeLa were supplemented with 4 L of Plus® Reagent, and BT-20 and HepG2 with 10 μL of Plus® Reagent. For each condition, the renilla luciferase reporter vector (100 ng) was always co-transfected to normalize for transfection efficiency. Cells were then lysed 24 h after transfection. Luciferase and renilla luciferase activities were measured using Dual-Luciferase® Reporter Assay System according to the manufacturer's protocol (Promega, Madison, Wis., USA) and a Lmax Luminometer (Molecular Devices, Sunnyvale, Calif., USA). Resulting luciferase activities were expressed in relative light units (RLU) and adjusted to the renilla luciferase activity. A positive control containing CMV promoter sequence (pGL3-Control) and a negative control (pGL3-Basic) were used for each experiment. Results, adjusted to the positive control, are representative of at least 3 independent experiments, expressed in fold of pGL3-Basic activity +/−standard error of the mean (SEM). When appropriate, the statistical significance of differences observed between luciferase activities was determined using the student t-test.

Co-transfection experiments using pOct1, pOct2 and peOct were performed as stated above except that 0.26 nM of the expression vector was co-transfected with 1.33 nM of the P4 construct.

### Results

Identification of the transcription initiation sites by rapid amplification of cDNA ends—The SBEM gene located on chromosome 12q13, spans a 3.9-kb long region consisting of 4 exons and 3 introns (FIGS. 2 and 4). The corresponding transcript, shown to be approximately 600-700 bp long, encodes a secreted protein of 90 amino acids (Miksicek et al., 2002; Colpitts et al., 2002). Sequence analysis of the 5′-flanking region of the SBEM gene revealed the presence of two putative overlapping TATA boxes in −100/−95 and −98/−93 (FIGS. 2 and 4) upstream from the translation start site (ATG). To precisely locate the beginning of the promoter region and to identify the exact transcription initiation site(s) of the SBEM gene, we performed a rapid amplification of 5′-cDNA ends followed by PCR amplification (RACE-PCR) on total RNA from 3 different breast cancer cell lines (MCF-7, T5 and MDA-MB-468) as described above. The RACE-PCR products were subsequently cloned and sequenced, allowing us to identify experimentally two distinct transcription initiation sites in −67 and −69 upstream of the ATG (FIGS. 2 and 4). RNAs initiated at both transcription initiation sites were found in all the three cell lines.

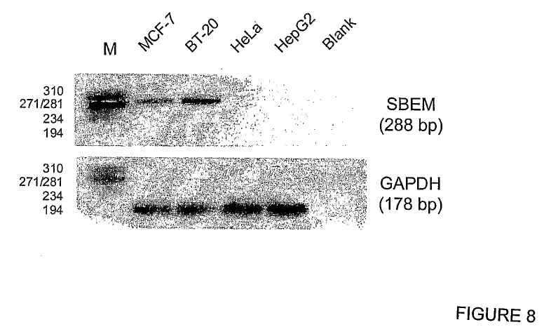

Endogenous SBEM promoter activity in mammary and non-mammary cancer cells—To further study the activity of the SBEM promoter in a mammary and a non-mammary context, we selected 2 breast (MCF-7, BT-20) and 2 non-breast (cervix: HeLa; liver: HepG2) cancer cell lines. We first assessed the activity of the endogenous SBEM promoter in these cell lines by investigating SBEM mRNA expression using reverse transcription followed by PCR amplification, as described above. As shown in FIG. 8, a single band of 288 by corresponding to SBEM mRNA was readily detected in both breast tumor but not in non-breast tumor cell lines. In contrast, the expression of the housekeeping gene GAPDH was uniform in all the cell lines analyzed, Identities of all PCR products were subsequently confirmed by sequencing and shown to correspond to the previously published sequences [GenBank: AF414087 and NM_{—}002046, for SBEM and GAPDH mRNA sequences, respectively].

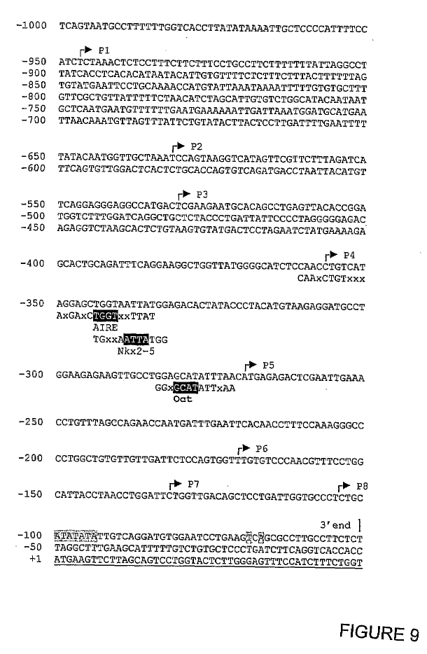

Analysis of SBEM promoter activity in mammary and non-mammary cancer cells—A series of promoter fragments fused to the coding region of the luciferase gene was constructed through successive deletions of the −900-bp 5′-region (−947/−51) as described above. The exact sequences used (P1-P8) are given in FIG. 9 whereas a schematic representation is shown in FIG. 5A. These constructs were then transfected into the mammary (MCF-7, BT-20) and non-mammary (HeLa and HepG2) cancer cells, and resulting luciferase activities were measured. As seen in FIG. 4B, the longest promoter construct P1, containing regions from −947 to −51 upstream of the ATG, led to a strong luciferase activity in both mammary cell lines. This activity of P1 construct ranged from 28 and 38 fold over the empty vector (the baseline control) in MCF-7 and BT-20, respectively. In contrast, this construct resulted in an activity lower than 4 fold the control in the two non-breast cells, HeLa and HepG2 (FIG. 5B), thus suggesting a breast-specific regulation of the SBEM promoter. Subsequent partial 5′-deletions (P2 and P3) did not change the promoter activity significantly in any of the cell lines analyzed. Interestingly, the removal of −531/−357 region (P4) led to a strong increase in the promoter activity in breast cells (124 and 95 fold for MCF-7 and BT-20, respectively), suggesting the presence of negative regulatory elements in the region encompassed between −531 and −357.

The P4 construct (−357/−51) had an 85-130 fold stronger promoter activity in mammary cells compared to non-mammary cells (FIG. 5B). The deletion of the subsequent-87-bp sequence (−357/−270) in the P5 construct reduced the promoter activity dramatically in the mammary cells (6.2 and 2.7 fold less than the P4 activity for MCF-7 and BT-20, respectively), suggesting the existence of a putative breast-specific enhancer region located within this 87-bp fragment. Further deletion of the region between −132 and −106 bp completely abolished the reporter activity (less than 1.2 fold the activity driven by the promoterless vector for all cells).

In these experiments, independently of the constructs used, promoter activity was always higher in mammary cells compared to non-mammary cells. Four different SBEM promoter regions were identified: from −531 to −357, which contains an apparent repressive activity; the 87-bp region from −357 to −270, which possesses an enhancer activity (ENH region), the −132/−51 region, which represents the minimal promoter, and the region from −947 to −51 containing the basal breast-specific activity conserved in the full-length promoter.

The ENH region (−357/−270) is able to drive a strong breast-specific promoter activity—In order to address the possible role of the ENH region (87-bp region between −357 and −270 upstream from the ATG) in SBEM promoter activity, two mutants, P1 plus and P1 minus, were constructed. P1 plus consisted of the basic P1 construct supplemented with an additional copy of the 87-bp region whereas P1 minus lacked this region (FIG. 6A). These constructs were then transfected in mammary and non-mammary cells, and luciferase activities were measured, as described previously. As shown in FIG. 6B, addition of a second ENH region to the full-length promoter (P1 plus) did not significantly modify the activity of the promoter in MCF-7 or BT-20 cells. However, deletion of this region (P1 minus) decreased the luciferase activity in all the mammary cells (from 28 to 9.5 and 38 to 4.7 fold the empty vector activity in MCF-7 and BT-20, respectively). As expected, addition or deletion of the 87-bp region to the full-length promoter did not significantly modify the luciferase activity in non-mammary cells (FIG. 6B).

It was surprising that while the removal of the 87-bp region strongly reduced the promoter activity, the addition of one extra copy of this region did not increase the transcriptional activity. As underlined earlier, our results suggested the existence of a repressor region (−531/−357), which might repress the enhancer activity of the 87-bp ENH region. To address this possibility, a P4plus mutant promoter was constructed, consisting of P4 supplemented with an additional copy of the ENH region (FIG. 7A). The activity of P4plus mutant was then compared to the activity observed with P4 (only one copy of the 87-bp region) and P5 (without this particular 87-bp sequence). As shown in FIG. 7B, the luciferase activity of the P4plus construct in all mammary cells was approximately twice the P4 activity, i.e. from 124 to 273 and 95 to 195 fold in MCF-7 and BT-20, respectively. This represented an average of a 10-fold increase compared to the P5 construct activity. Interestingly, this construct still remained free of any luciferase activity in the non-mammary cells.

Importance of octamer-binding transcription factors motif in the SBEM promoter activity—In order to further identify potential sequences involved in the strong enhancer effect of the 87-bp region on the reporter gene activity, we searched for putative transcription factor binding sites, using Matinspector software as discussed above. As shown in FIG. 9, only 3 different motifs were identified within this region. The first two motifs overlapped in a region located between −361 and −335 and consisted of binding sites for AIRE (autoimmune regulator) and Nkx2-5 (cardiac-specific homeobox protein NK-2 homolog E). The third, located in −284/−272, corresponded to an octamer-binding transcription factor site (Oct¹ motif).

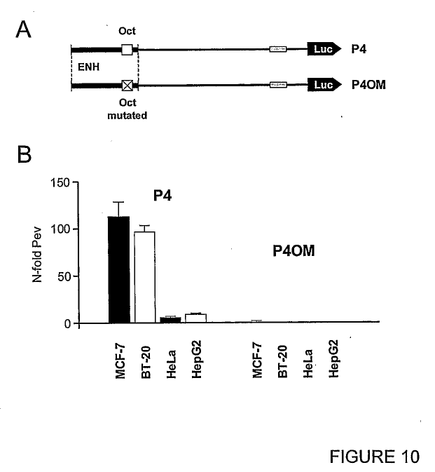

To determine whether the octamer binding site participated to the strong breast expression of the reporter gene, an Oct-mutated SBEM promoter construct (P40M) was generated, by substituting the octamer-binding site GGAGCATATTTAA (SEQ ID No. 34) located in −284/−272 by GGTCTAATGTAAA (SEQ ID No. 35). P4 and P40M were transiently transfected in mammary and non-mammary cell lines, and luciferase activities were measured as stated above. As shown in FIG. 10, mutation of the octamer-binding site in P40M totally abolished the luciferase activity in MCF-7 and BT-20 (from 111 and 97 to less than 1 fold, respectively). Despite the fact that P4 activity was already extremely low in non-mammary cell lines, the mutation of the octamer-binding site nonetheless led to a further decrease of the luciferase activity.

Oct1 and Oct2 are able to enhance both exogenous and endogenous SBEM promoter activities—In order to determine the potential role of the Oct transcription factors in the regulation of the SBEM promoter activity, the P4 construct was co-transfected with pOct1 and pOct2 expression vectors, or the empty vector as discussed above. Luciferase activity was then measured 24 hours following transfection as described above.

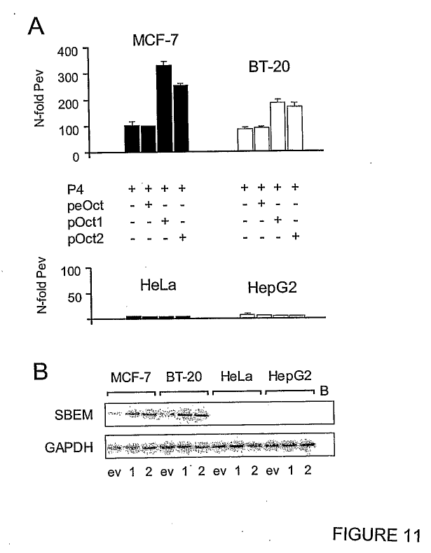

Co-transfection with the empty vector (peOct) did not alter the luciferase activity of the P4 construct (FIG. 11A). However, transient over-expression of either Oct1 or Oct2 transcription factors in mammary cell lines led to an increase in the P4 luciferase activity. In MCF-7, an increase from 100 to 342 and 100 to 264 fold was observed following Oct1 and Oct2 over-expression, respectively. Similarly, the increase in BT-20 was from 93 to 191 and from 93 to 173 fold following Oct1 and Oct2 over-expression, respectively. This corresponded to an average of a 2.5 fold increase compared to the P4 activity co-transfected with the corresponding empty vector. Over-expression of Oct1 and Oct2 transcription factors did not modify the luciferase activity in the non-mammary cells.

To investigate a putative role of Oct1 and Oct2 on the regulation of the endogenous SBEM gene expression, total RNA was extracted from both mammary and non-mammary cells 24 hours following the transfection with pOct1, pOct2 or the empty expression vector. Transcripts were then reverse transcribed and PCR amplified using SBEM and GAPDH primers as described above. As shown in FIG. 11B, endogenous SBEM gene expression was up-regulated in mammary cell lines following Oct1 and Oct2 over-expression. In contrast, the GAPDH gene expression was uniform in all conditions, and no variation in the SBEM gene expression was observed in non-breast cancer cells.

While the preferred embodiments of the invention have been described above, it will be recognized and understood that various modifications may be made therein, and the appended claims are intended to cover all such modifications which may fall within the spirit and scope of the invention.

TABLE 1

| | | | |
|---|---|---|---|
| Name | Sequence | Position | SEQ ID |

| | | | |
|---|---|---|---|
| SBEM-F | GATCTTCAGGTCACCACCATG | −18/+2 | 12 |
| SBEM-R | GGGACACACTCTACCATTCG | +251/+270 | 13 |
| GAP-F | ACCCACTCCTCCACCTTTG | +868/+886 | 14 |
| GAP-R | CTCTTGTGCTCTTGCTGGG | +1027/+1045 | 15 |
| RACE-R1 | CAGAAGACTCAAGCTGATTCC | +277/+297 | 16 |
| RACE-L2 | TCTTTACGAGCAGTGGTAGAA | +214/+234 | 17 |
| Adap-F1 | CTAATACGACTCACTATAGGGC | | 18 |
| Adap-F2 | AAGCAGTGGTATCAACGCAGAGT | | 19 |
| P947-F | GCTCCCCATTTTCCATCTCGAGCATC | −964/−939 | 20 |
| P632-F | CAATGGTTGCTAACTCGAGTAAGGTC | −646/−621 | 21 |
| P531-F | AGGGAGGCCATGACTCGAGGAATG | −545/−522 | 22 |
| P357-F | CTCCAACTCGAGATAGGAGCTGG | −364/−342 | 23 |
| P270-F | GGAGCATATTTAACTCGAGAGACTCG | −284/−259 | 24 |
| P170-F | CTCCAGTGGCTCGAGTCCCAACGTT | −181/−157 | 25 |
| P132-F | TAACCTGGATCTCGAGTGACAGCTCC | −143/−118 | 26 |
| P106-F | CCTGATTGGTGCCTCGAGCATATATATTGTC | −119/−89 | 27 |
| P51-R | CTTCAAAGCCTAAGCTTAGGCAAGGCGC | −66/−39 | 28 |
| P270nde-F | CATATTTAACATATGAGACTCCAATTGAAACCTG | | 29 |
| P357nde-R | GGGGCATCTCCCAACATATGATAGGAGC | | 30 |
| 87kpn-F | (AC)₅-GGTACCTCATAGGAGCTGGTAATTATGG | | 31 |
| 87xho-R | GAAGTTGCCTGGAGCATATTTAACCTCGAG-(GGTT)₃ | | 32 |
| P40M | GTTGCCTGGTCTAATGTAAACATGAGAGACTCG | | 33 |

1. A nucleic acid molecule having at least 70% homology to SEQ ID No. 2.

2. The nucleic acid molecule according to claim 1 having at least 70% homology to SEQ ID No. 3.

3. The nucleic acid molecule according to claim 1 having at least 70% homology to SEQ ID No. 4.

4. The nucleic acid molecule according to claim 1 having at least 70% homology to SEQ ID No. 5.

5. The nucleic acid molecule according to claim 1 having at least 70% homology to SEQ ID No. 7.

6. The nucleic acid molecule according to claim 1 having at least 70% homology to SEQ ID No. 10.

7. The nucleic acid molecule according to claim 1 operably linked to a gene of interest.

8. The nucleic acid molecule according to claim 1 operably linked to a cytotoxic gene or an antitumor gene.
